(19) United States
(12) Patent Application Publication
(10) Pub. No.: US 2025/0072944 A1
(43) Pub. Date: Mar. 6, 2025

(54) BONE FIXATION ASSEMBLY

(71) Applicant: LIANG-CHENG CHEN, Taichung City (TW)

(72) Inventor: LIANG-CHENG CHEN, Taichung City (TW)

(21) Appl. No.: 18/529,374

(22) Filed: Dec. 5, 2023

(30) Foreign Application Priority Data
Sep. 6, 2023 (TW) 112133932

(51) Int. Cl. A61B 17/80; A61B 17/86

(52) U.S. Cl. CPC A61B 17/8071; A61B 17/86

(57) ABSTRACT

A bone fixation assembly includes a bone plate, a first bone screw and a second bone screw. The first and second bone screws penetrate through two holes respectively. The first bone screw has a left-handed thread while the second bone screw has a right-handed thread. With different thread directions of the bone screws, the bone screws are hard to be loosened so that broken bones are fixed firmly. Accordingly, size of the bone plate is able to be reduced for miniaturization.

### BACKGROUND OF INVENTION

### 1. Field of Invention

The present invention relates to medical equipment and, more particularly, to bone plates and bone screws.

### 2. Related Prior Art

Bone fractures as a result of accidental impact or surgical operation requires the securing of the broken bones during recovery to help with healing process. The conventional method of fixation is by applying plaster externally which solidifies into a rigid shell that restricts the movement of limb. Although this method is relatively simple to implement, it nevertheless causes great inconvenience to patients, and thus has been gradually replaced by internal fixation method.

The so called internal fixation method refers to using a surgical method to bridge a bone plate between two broken bones, and by inserting bone screws through a bone plate into the two broken bones, so that the two broken bones are secured by the bone plate. Unlike the aforementioned external fixation method, this method does not impose a burden on the patient's daily, and provides better fixation and accelerate the healing process. It is known that the longer the bone plate, the more bone screws can be inserted; and the thicker the bone plate, the less it is prone to deformation, providing better support. While both are able to enhance the fixation of the bones, the drawback is an increased discomfort for the patient. In certain circumstances, such as limited space of the affected area or in close proximity to structures such as nerves or blood vessels, an excessive long or thick bone plates cannot be used. On the other hand, placing a larger bone plate requires a large surgical incision, which is accompanied by greater pain and an extended healing period. Therefore, in this case, a large bone plate is not necessarily better.

In view of the above, given a sufficient bone fixation, the choice of the bone plate is naturally the smaller, the better. In light of the current surgical technique and practical experience, at least two bone screws need to be applied between the bone plate and one side of the broken bone to stabilize the bones firmly, and the entire bone fixation device requires four bone screws and a bone plate that could accommodate the four bone screws. If the broken bone is located in a relatively spacious area, such as the hand or leg, there is not much of an issue. However, in the case of orthognathic surgery, it can be challenging to insert four bone screws, and the stability effect suffers if only two bone screws are applied to the cut mandible, causing the bones to move, resulting in poor healing.

### SUMMARY OF INVENTION

The present invention provides a bone fixation assembly composed of a single bone plate, a single first bone screw and a single second bone screw. The bone plate is provided with two through holes. The first bone screw is provided with a left-handed thread to be screwed into a bone via one of the through holes. The second bone screw is provided with a right-handed thread to be screwed into another bone via the other one of the through holes.

In one embodiment, the bone plate includes an extended portion located between the two through holes, the extended portion having an additional through hole.

In another embodiment, the bone plate includes an extended portion located between the two through holes, the extended portion having no through hole.

Preferably, each of the through holes is provided with a thread fitted with the corresponding bone screw.

The present invention further provides a bone fixation assembly includes a bone plate with at least four through holes and at least four bone screws. Each of the bone screws is screwed into bones via the through holes. At least one bone screw is provided with a thread in opposite direction to the other bone screws.

Preferably, the bone plate includes a left portion, a right portion and an extended portion between the left portion and the right portion. Half through holes are disposed on the left portion while other through holes are disposed on the right portion. The extended portion has no through hole.

Preferably, the thread of any one of the bone screws is in opposite direction to adjacent bone screws.

Furthermore, each of the through holes is provided with a thread fitted with the corresponding bone screw.

Other objectives, advantages and features of the present invention will be apparent from the following description referring to the attached drawings.

### BRIEF DESCRIPTION OF DRAWINGS

The present invention will be described via detailed illustration of the preferred embodiment referring to the drawings wherein:

FIG. 1 is a perspective exploded view of a first embodiment of the present invention;

FIG. 2 is a perspective view of a bone plate according to another type of the first embodiment of the present invention;

FIG. 3 illustrates how the first embodiment of the present invention is used in orthognathic surgery;

FIG. 4 is a perspective exploded view of a second embodiment of the present invention; and

FIG. 5 is a perspective view of a bone plate according to another type of the second embodiment of the present invention.

### DETAILED DESCRIPTION OF PREFERRED EMBODIMENT

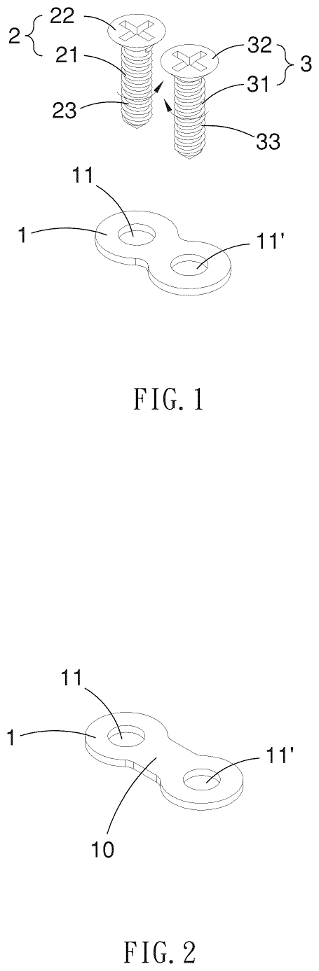

FIG. 1 shows a first embodiment of a bone fixation assembly of the present invention composed of one bone plate 1, one first bone screw 2 and one second bone screw 3. The shape of the bone plate 1 is not limited to what is shown in the figure. Two spaced through holes 11, 11′ are provided on the bone plate 1. As another type shown in FIG. 2, the bone plate 1 extends in a longitudinal direction and has an extended portion 10 disposed between two through holes 11, 11′. One or more additional through holes may be provided on the extended portion 10, or there is no through hole on the extended portion 10.

The first bone screw 2 includes a body 21 with a left-handed thread 23 and a head 22 with a greater diameter than that of the through holes 11, 11′. The second bone screw 3 includes a body 31 with a right-handed thread 33 and a head 32 with a greater diameter than that of the through holes 11, 11′.

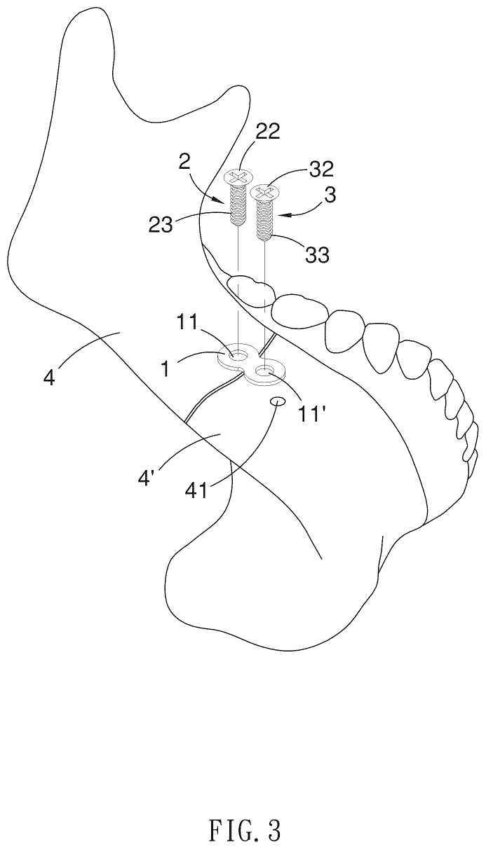

Referring to FIG. 3, the bone plate 1 is used to be bridged between two broken bones, which means one through hole 11 is located on one bone 4 while the other through hole 11′ is located on the other bone 4′. The first bone screw 2 penetrates through the through hole 11 to be screwed into the bone 4, and the second bone screw 3 penetrates through the through hole 11′ to be screwed into the bone 4′. Both the head 22, 32 press upon the bone plate 1 to fix the bone plate 1 on the bone 4, 4′. The first bone screw 2, based on the left-handed thread 23, needs counter-clockwise rotation to be screwed into the bone 4. On the other hand, the second bone screw 3, based on the right-handed thread 33, needs clockwise rotation to be screwed into the bone 4′. In this embodiment, the through holes 11, 11′ may have a thread fitted with the corresponding bone screw.

According to the present invention above-mentioned, even though one of the bone screws is loosened by vibrations produced in patient's daily life, the other bone screw is still firmly fixed as a result of different thread directions of the two bone screws 2, 3. In other words, this ensures that at least one bone screw is not loosened when two bone screws with different thread directions face vibration. Comparing to the conventional bone fixation assembly which requires two bone screws screwed into one bone (four bone screws for two bones), the present invention only requires one bone screw screwed into one bone (two bone screws for two bones) to satisfy fastening strength for good healing, and therefore area of the bone plate 1 can be reduced for miniaturization. In addition to narrowing the surgical incision and reducing discomfort, the present invention is particularly suitable for orthognathic surgery which is performed on cut mandible in narrow oral environments as shown in FIG. 3.

When using the bone plate 1 to fix the cut mandible, benefitting from the miniaturization, the bone plate 1 is kept away from the mental foramen 41 to avoid pressing the mental nerve, reducing the adverse consequences of nerve damage or facial paralysis.

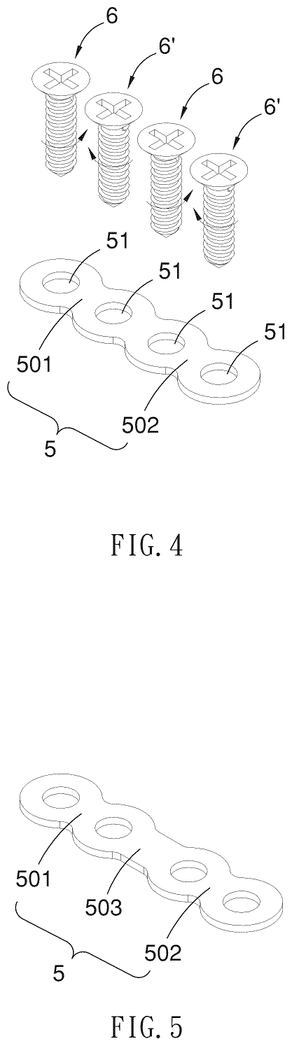

Referring to FIG. 4, a second embodiment of the present invention includes a bone plate 5 and four bone screws 6, 6′. The bone plate 5 is provided with four through holes 51. Furthermore, the bone plate 5 defines a left portion 501 and a right portion 502, each of them having two through holes 51. The bone plate 5 may include an extended portion 503 between the left portion 501 and the right portion 502 as shown in FIG. 5 for arrangement for positions of the through holes 51. There is no through hole on the extended portion 503.

Among the above-mentioned four bone screws 6, 6′, at least one bone screw is provided with a thread in opposite direction to the other bone screws. In this embodiment, the thread of anyone of the bone screws 6, 6′ is in opposite direction to adjacent bone screws. For example, each bone screw 6 has a left-handed thread while each bone screw 6′ has a right-handed thread. Each of the through holes 51 may have a thread fitted with the corresponding bone screw.

The more bone screws than that of the first embodiment not only raise fastening strength for broken bones due to amount of the bone screws but also avoid being loosened concurrently because of different thread directions of the bone screws.

What is claimed is:

1. A bone fixation assembly composed of a single bone plate, a single first bone screw and a single second bone screw, wherein the bone plate is provided with two through holes, wherein the first bone screw is provided with a left-handed thread and is configured to penetrate through one of the through holes, wherein the second bone screw is provided with a right-handed thread and is configured to penetrate through the other one of the through holes.

2. The bone fixation assembly according to claim 1, wherein the bone plate includes an extended portion located between the two through holes, the extended portion having an additional through hole.

3. The bone fixation assembly according to claim 1, wherein the bone plate includes an extended portion located between the two through holes, the extended portion having no through hole.

4. The bone fixation assembly according to claim 1, wherein each of the through holes is provided with a thread fitted with the corresponding bone screw.

5. A bone fixation assembly comprising:
a bone plate with at least four through holes; and
at least four bone screws each configured to penetrate through the through holes, wherein at least one bone screw is provided with a thread in opposite direction to the other bone screws.

6. The bone fixation assembly according to claim 4, wherein the bone plate includes a left portion, a right portion and an extended portion between the left portion and the right portion, half through holes being disposed on the left portion while other through holes being disposed on the right portion, the extended portion having no through hole.

7. The bone fixation assembly according to claim 4, wherein the thread of any one of the bone screws is in opposite direction to adjacent bone screws.

8. The bone fixation assembly according to claim 4, wherein each of the through holes is provided with a thread fitted with the corresponding bone screw.
